(19) United States
(12) Patent Application Publication
(10) Pub. No.: US 2024/0398263 A1
(43) Pub. Date: Dec. 5, 2024

(54) FORECASTING BLOOD GLUCOSE CONCENTRATION

(71) Applicant: INFORMED DATA SYSTEMS INC. d/b/a ONE DROP, New York, NY (US)

(72) Inventors: Daniel R. Goldner, Minnetonka, MN (US); Jeffrey Dachis, Brooklyn, NY (US)

(21) Appl. No.: 18/426,190

(22) Filed: Jan. 29, 2024

Related U.S. Application Data
(63) Continuation of application No. 16/558,558, filed on Sep. 3, 2019, now abandoned.
(60) Provisional application No. 62/854,088, filed on May 29, 2019.
(60) Provisional application No. 62/728,496, filed on Sep. 7, 2018.

(51) Int. Cl. A61B 5/145; G06N 20/00; G16H 50/20

(52) U.S. Cl. CPC A61B 5/14532; G06N 20/00; G16H 50/20

(57) ABSTRACT

A method, a system and a computer program product for forecasting blood glucose concentration. One or more features for training a blood glucose concentration forecasting model are determined. The features are determined based on one or more input data parameters associated with a user in a plurality of users. Using the determined one or more features, the blood glucose concentration forecasting model is trained. Using the trained blood glucose concentration forecasting model, one or more expected blood glucose concentrations for the user are generated.

### CROSS-REFERENCE TO RELATED APPLICATIONS

The present application is a continuation of U.S. application Ser. No. 16/558,558, filed Sep. 3, 2019, which claims priority to U.S. Provisional Patent Appl. No. 62/728,496, filed Sep. 7, 2018, and entitled “Forecasting Blood Glucose Concentration” and U.S. Provisional Patent Appl. No. 62/854,088, filed May 29, 2019, and entitled “Forecasting Blood Glucose Concentration,” and the present application incorporates their disclosures herein by reference in their entireties.

### TECHNICAL FIELD

This disclosure relates generally to data processing and, in particular, to forecasting blood glucose concentration and/or interpreting forecasted data.

### BACKGROUND

Diabetes mellitus (DM) is a group of metabolic disorders in which there are high blood sugar levels over a prolonged period. Typical symptoms of such condition include frequent urination, increased thirst, increased hunger, etc. If left untreated, diabetes can cause many complications. There are three main types of diabetes: Type 1 diabetes results from pancreas's failure to produce enough insulin. In Type 2 diabetes, cells fail to respond to insulin properly. Gestational diabetes occurs when pregnant women without a previous history of diabetes develop high blood sugar levels.

Diabetes affects a significant percentage of world's population. Timely and proper diagnoses and treatment are essential to maintaining relatively healthy lifestyle for individuals with diabetes. Application of treatment typically relies on accurate determination of glucose concentration in the blood of an individual at a present time and/or in the future. Conventional systems are unable to provide accurate prediction or forecast of blood glucose concentration at a point in time in the future. Thus, there is a need for a system and a method that is capable of accurately forecasting blood glucose concentration for an individual that is based on the information/data about that individual and/or other similarly situated individuals.

### SUMMARY

In some implementations, the current subject matter relates to a computer implemented method for forecasting blood glucose concentration. The method can include determining one or more features for training a blood glucose concentration forecasting model, wherein the one or more features are determined based on one or more input data parameters associated with a user in a plurality of users, training, using the determined one or more features, the blood glucose concentration forecasting model, and generating, using the trained blood glucose concentration forecasting model, one or more expected blood glucose concentrations for the user.

In some implementations, the current subject matter can include one or more of the following optional features. The method can further include displaying the generated expected blood glucose concentrations for the user on one or more graphical user interfaces.

In some implementations, the training can include training the blood glucose concentration forecasting model using one or more parameters associated with one or more other users in the plurality of users. The parameters associated with other users can include one or more historical data parameters associated with one or more other users.

In some implementations, the input parameters can include at least one of the following: a data indicating a type of diabetes of the user, a data indicating a medical condition of the user, a data indicating medication being consumed by the user, a data indicating a meal consumed by the user, a data indicating a physical activity performed by the user, a data indicating a time of a blood glucose concentration measurement of the user, a data indicating at least one of a previous and a current value of a blood glucose concentration measurement of the user, a data indicating a time of a previous blood glucose concentration forecast, a data indicating a target blood glucose concentration (a1c) for the user, a data indicating at least one of a current date and a current time, a data indicating a weight of the user, a data indicating one or more changes in the blood glucose concentration of the user, a data indicating one or more carbohydrate values as consumed by the user, and any combination thereof.

In some implementations, the generating can include generating one or more target blood glucose concentration ranges for the user, generating one or more confidence intervals for the generated expected blood glucose concentrations, where the confidence intervals can indicate an accuracy of the generated one or more expected blood glucose concentrations, and comparing the generated target blood glucose concentration ranges, the confidence intervals for the generated expected blood glucose concentrations, and the generated expected blood glucose concentrations. The method can also include displaying, based on the comparison, an indication whether the generated expected blood glucose concentrations is within the target blood glucose concentration ranges. The method can further include generating an alert to the user when the generated expected blood glucose concentrations is not within the target blood glucose concentration ranges.

In some implementations, the generated expected blood glucose concentrations can be generated for a point in time subsequent to the determining.

In some implementations, the method can also include repeating the determination of the features, as well as the training of the forecasting model, and then, generating, based on the repeated determinations and training, one or more updated expected blood glucose concentrations for the user.

In some implementations, the current subject matter relates to a computer implemented method for forecasting and interpreting blood glucose concentration for a user. The method can include determining features (e.g., input data parameters) for training a forecasting data model, training the model, generating blood glucose concentration predictions, determining confidence intervals for the predictions, generating target ranges for the blood glucose concentration values, combining forecasting data, confidence intervals and target ranges for display to a user, and interpreting the forecasted data, e.g., to provide feedback to the user.

In some exemplary implementations, the current subject matter can provide a method for determining forecasts of a user's blood glucose concentration (BG) at a point in the future from 15-minutes to 24-hours, quantifying confidence intervals relating to the forecasted data, and producing an interpretation of whether the forecast is above, below or within the range consistent with any given target blood glucose health (a1c) goal. For forecasting purposes, the current subject matter can use past blood glucose concentration values, grams carbohydrates eaten at meals, workouts or minutes of activity, past values of weight, past values of a1c, year of diagnosis, etc., and/or any combination thereof. It can also use the above information that users have entered, which can widely vary from user to user, and from month to month for a given user.

In some exemplary implementations, the current subject matter can organize the above information to make it amenable to machine learning (e.g., “feature engineering”), whereby irregular and/or gappy historical information can be transformed into a standard form for each prediction being made. This can allow the model to make predictions for one user based on histories of other users in similar situations. In some exemplary, non-limiting implementations, the current subject matter can predict blood glucose in advance for pre-diabetes, gestational diabetes, Type 2 diabetes that are using no insulin and/or using basal insulin and/or using bolus insulin, with 75% of “test set” predictions within 34 mg/dL and 86% of predictions within 50 mg/dL. The accuracy of the above model is based on not only the user's particular information, but also from the information obtained from other users. The current subject matter can also provide confidence intervals, e.g., how close the forecasted data is close to real values. For example, for a particular user, two hours from now, the current subject matter can be
50% confident that the actual blood glucose concentration will be between 128-152mg/dL;
75% confident it will be between 120-158 mg/dL;
90% confident it will be between 90-170 mg/dL.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1a illustrates an exemplary system for forecasting and interpreting blood glucose concentration data, according to some implementations of the current subject matter;

FIG. 1b illustrates an exemplary forecasting and analysis engine shown in FIG. 1a, according to some implementations of the current subject matter;

FIG. 1c illustrates an exemplary process that performed by the forecasting and analysis engine shown in FIG. 1b, according to some implementations of the current subject matter;

FIGS. 2a and 2b illustrate an exemplary data/information that may be provided to/used by the system shown in FIG. 1a, according to some implementations of the current subject matter;

FIG. 3 is a plot containing exemplary short-term blood glucose forecasts (in mg/dL over time) generated by the system shown in FIG. 1a, according to some implementations of the current subject matter;

FIG. 4 illustrates an exemplary process that can be performed by the system shown in FIG. 1a for forecasting blood glucose concentration, according to some implementations of the current subject matter;

FIGS. 5a-b illustrate tables containing exemplary non-limiting training model inputs (e.g., local hour, local day of week, etc.), according to some implementations of the current subject matter;

FIG. 6 is a plot illustrating exemplary confidence intervals for a training set and a prediction set (i.e., close correspondence of the training and test set quantiles), according to some implementations of the current subject matter.

FIG. 7 illustrates a table containing exemplary target ranges, according to some implementations of the current subject matter;

FIG. 8 is a plot showing exemplary target blood glucose concentration values, according to some implementations of the current subject matter;

FIG. 9 illustrates an exemplary user interface, according to some implementations of the current subject matter;

FIG. 10 is an exemplary forecasting plot, according to some implementations of the current subject matter;

FIGS. 11a-d illustrate various exemplary graphical user interfaces that may be generated by the current subject matter system shown in FIG. 1a, according to some implementations of the current subject matter;

FIGS. 12a-b illustrate exemplary user interfaces that may be used by the user to customize information that is being displayed to the user, according to some implementations of the current subject matter;

FIG. 13 illustrates an exemplary system, according to some implementations of the current subject matter;

FIG. 14 illustrates an exemplary method, according to some implementations of the current subject matter

### DETAILED DESCRIPTION

To address these and potentially other deficiencies of currently available solutions, one or more implementations of the current subject matter relate to methods, systems, articles of manufacture, and the like that can, among other possible advantages, provide a way to forecast and interpret blood glucose data and other data related to the user.

In some implementations, the current subject matter can provide a computing system and/or framework for performing such determining, forecasting and/or interpretation of blood glucose data and other data related to the user. The data can include data, metadata, structured content data, unstructured content data, embedded data, nested data, hard disk data, memory card data, cellular telephone memory data, smartphone memory data, main memory images and/or data, forensic containers, zip files, files, memory images, and/or any other data/information. The input and/or the output data can be in various formats, such as text, numerical, alpha-numerical, hierarchically arranged data, table data, email messages, text files, video, audio, graphics, etc. The input data can include at least one of the following: current and/or previous blood glucose measurement data of the user, current and/or previous blood glucose measurement data of other users (e.g., the data can be appropriately anonymized), meal characteristics data (e.g., number of meals, time of meals, grams carbohydrates consumed during meal times (whether currently and/or in the past)), physical exercise data (e.g., workout times, activity type (e.g., walking, running, etc.), current and/or previous weight data of the user, current and/or previous a1c data values, medical history data related to the user (e.g., family history, user health history, diagnoses, blood pressure, etc.), as well as similar type of data related to other users.

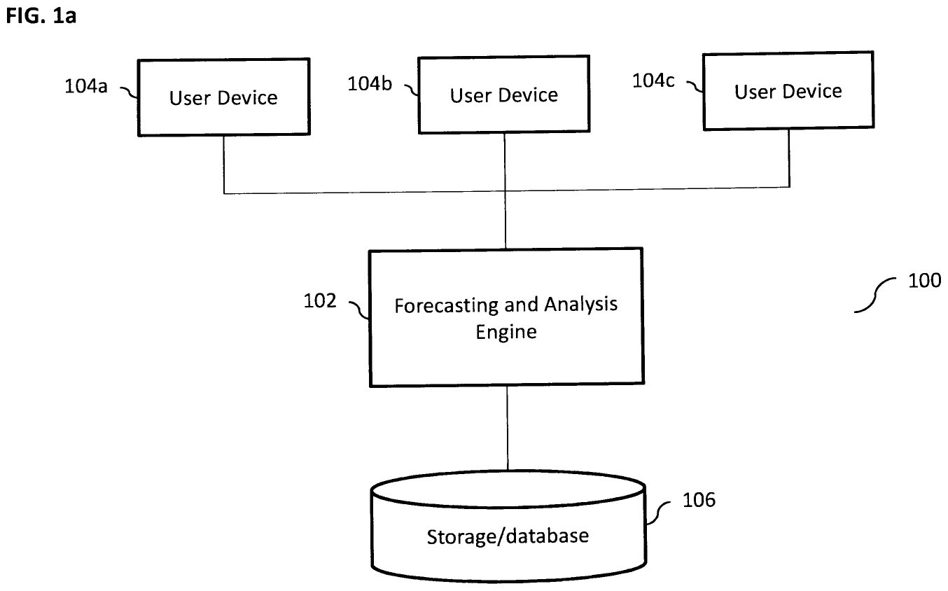

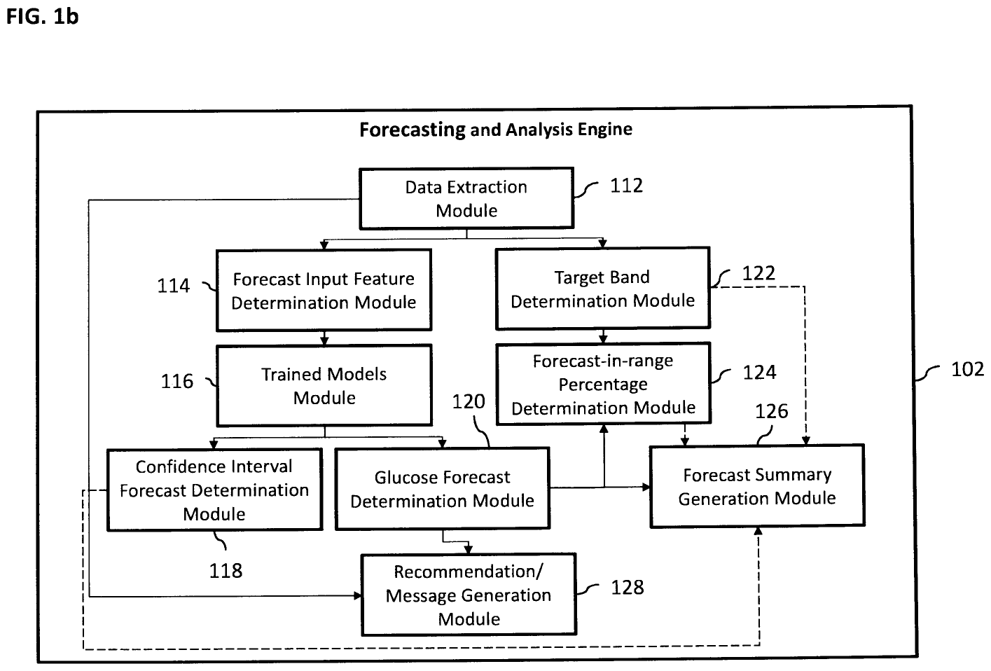

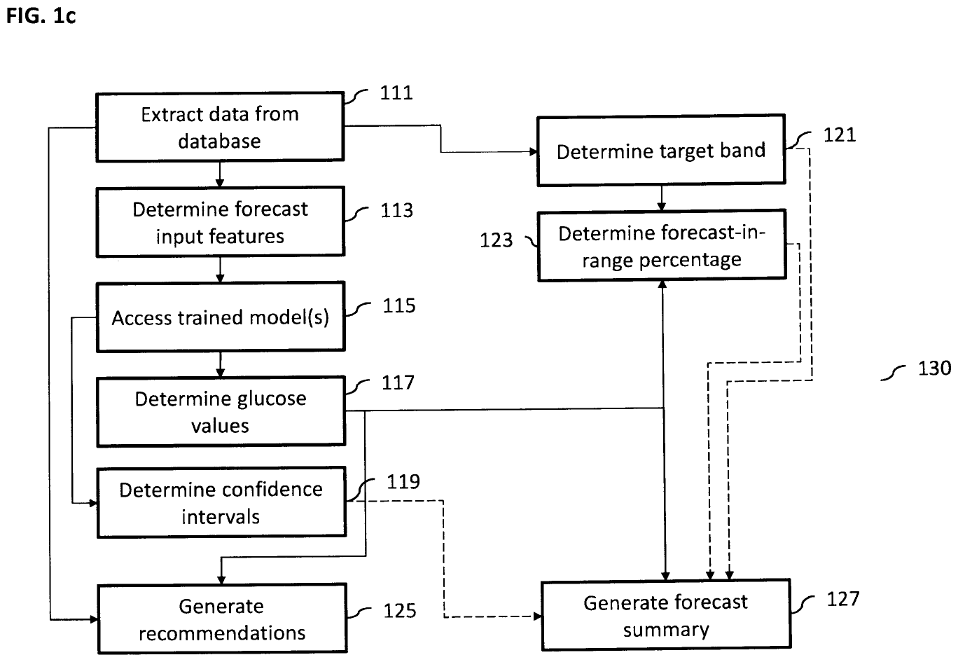

FIG. 1a illustrates an exemplary system 100 for performing determination, analysis, forecasting, interpretation, etc. of blood glucose measurements and/or any other data, according to some implementations of the current subject matter. The system 100 can include a forecasting and/or analysis engine and/or computing platform 102, one or more user devices 104(a, b, c), and a storage/database component 106. Components 102-106 can be communicatively coupled using one or more communications networks. The communications networks can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof.

The components 102-106 can include any combination of hardware and/or software. In some implementations, components 102-106 can be disposed on one or more computing devices, such as, server(s), database(s), personal computer(s), laptop(s), cellular telephone(s), smartphone(s), tablet computer(s), and/or any other computing devices and/or any combination thereof. In some implementations, the components 102-106 can be disposed on a single computing device and/or can be part of a single communications network. Alternatively, the components can be separately located from one another.

A user can access the system 100 via a user device 104. The user device 104 can be used to obtain blood glucose measurement data and/or any other data (e.g., health data, nutrition data, exercise data, etc.) relating to the user and/or any other users (appropriately anonymized). In some exemplary implementations, the user device 104 can include a component that is capable of obtaining blood samples from the user and determining glucose concentration levels in user's blood. Any means of obtaining blood samples from the user and/or determining blood glucose concentration levels can be used. The device can also include one or more data input components that can allow entry of various data (e.g., nutrition data (e.g., consumption times, number of calories, amount of fat, sugars, etc.), health data (e.g., weight, age, sleeping patterns, medical conditions, cholesterol levels, etc.), exercise data (e.g., walking, running, swimming, etc.), personal data (e.g., name, gender, social network information, etc.), and/or any other data, and/or any combination thereof), etc. In some implementations, the data can be queried by the user device 104 from one or more third party databases. The user device 104 can be used to generate a query and transmit it to the engine 102, which can determine which database may contain requisite information and then connect with that database to execute a query and retrieve appropriate information. In some implementations, the engine 102 can include various application programming interfaces (APIs) and/or communication interfaces that can allow interfacing between user devices 104, databases, and/or any other components.

As shown in FIG. 1a, one or more users using devices 104 can access the system 100. The users can be individual users, computing devices, software applications, objects, functions, and/or any other types of users and/or any combination thereof. Upon obtaining appropriate data (e.g., blood glucose measurement data, health data, etc., as discussed above), the user device 104 can generate an instruction/command to the engine 102 to process the obtained data and/or extract additional data from one or more databases component 106, and perform analysis of gathered and/or received data. In some implementations, the instruction/command can include an authentication token that may be used by the engine 102 to authenticate the user device 104 (e.g., it can be a passcode, a one-time/single-use generated number sequence, etc.). The instruction/command can be in a form of a query, a function call, and/or any other type of instruction/command. In some implementations, the instructions/commands can be provided using a microphone (either a separate microphone or a microphone imbedded in the user's computing device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the engine 102 to perform an analysis of the data that may exist in the system 100 (e.g., stored in the database 106) and/or inputted via the data device 104. The analysis can implement various machine learning techniques, as further discussed below.

Any means can be used to obtain data for the purposes of the analysis performed by engine 102, where the means can include one or more of the following a microphone (either a separate microphone or a microphone imbedded in the user device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The engine 102 can also obtain data from various third party sources. In some implementations, engine 102 can be communicatively coupled to various public and/or private databases that can store various information, e.g., census information, health statistics (appropriately anonymized), demographic information, population information, and/or any other information. For example, the engine 102 can be used to obtain information about blood glucose measurements/concentration and/or forecasts of blood glucose concentrations of a plurality of users (without identifying the users) of the system 100, nutrition data relating to such users, exercise data, social network information, and/or any other information and/or any combination thereof.

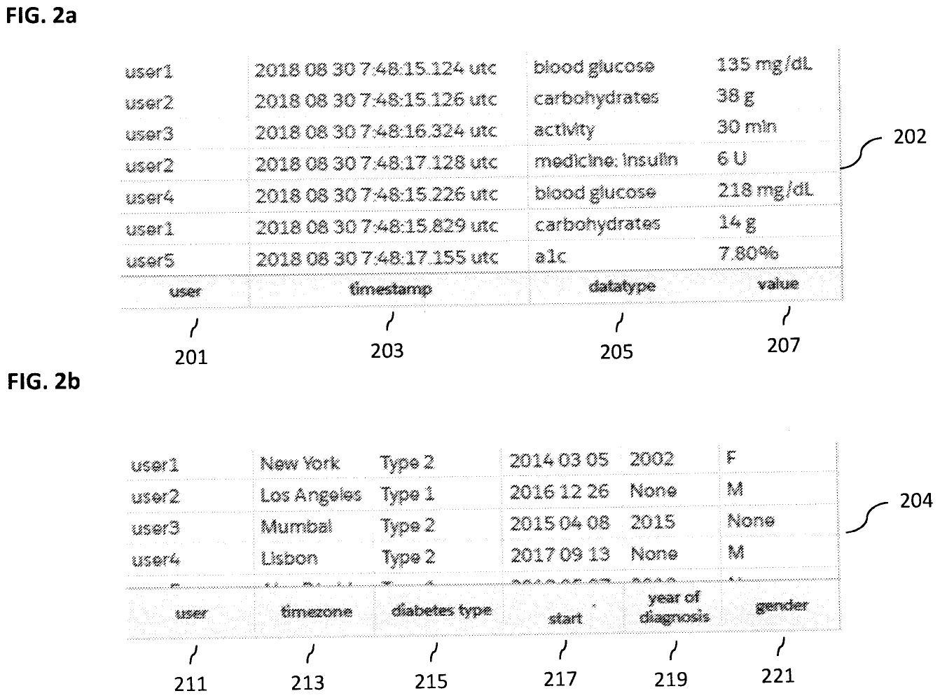

The engine 102 can execute a query that may be and/or process data received from the user device 104 and access the database 106 to obtain relevant data that may be stored in the database 106. Exemplary relevant data is shown in FIGS. 2a-b. In some implementations, the relevant data can be obtained or “fetched” for a specific user (e.g., “user1”, “user2”, etc. as shown in FIG. 2a-b). The engine 102 can obtain such relevant data upon the user entering new data (e.g., new user measurement data, new user weight data, new meal data, etc.). Alternatively, the engine 102 can obtain the above data upon a specific request (e.g., user navigating to a forecast screen on the user device 104, etc.) and/or query that may be generated to the engine 102 (e.g., by the user device 104, a third party application, etc.). The data can include data related to that user and/or a plurality of users (e.g., historical blood glucose concentration levels, prior analyses of blood glucose measurements, health history data, medical condition history data, exercise history data, nutrition data, etc.). The data can be appropriately anonymized to ensure compliance with various privacy standards. The database 106 can store information in various formats, e.g., table format, column-row format, key-value format, etc. (e.g., each key can be indicative of various attributes associated with the user and each corresponding value can be indicative of attribute's value (e.g., measurement, time, etc.)).

FIG. 1b illustrates exemplary components of the engine 102, according to some implementations of the current subject matter. FIG. 1c illustrates an exemplary method 130 that can be performed by the engine 102 for generation of predictions of blood glucose concentrations, according to some implementations of the current subject matter. The engine 102 can include one or more computing modules, functionalities, etc. that may be used to determine/forecast one or more blood glucose concentrations and/or generate one or more recommendations, explanation of forecasts, support messages, etc. As stated above, the engine 102 can include one or more hardware components, software components, and/or a combination thereof.

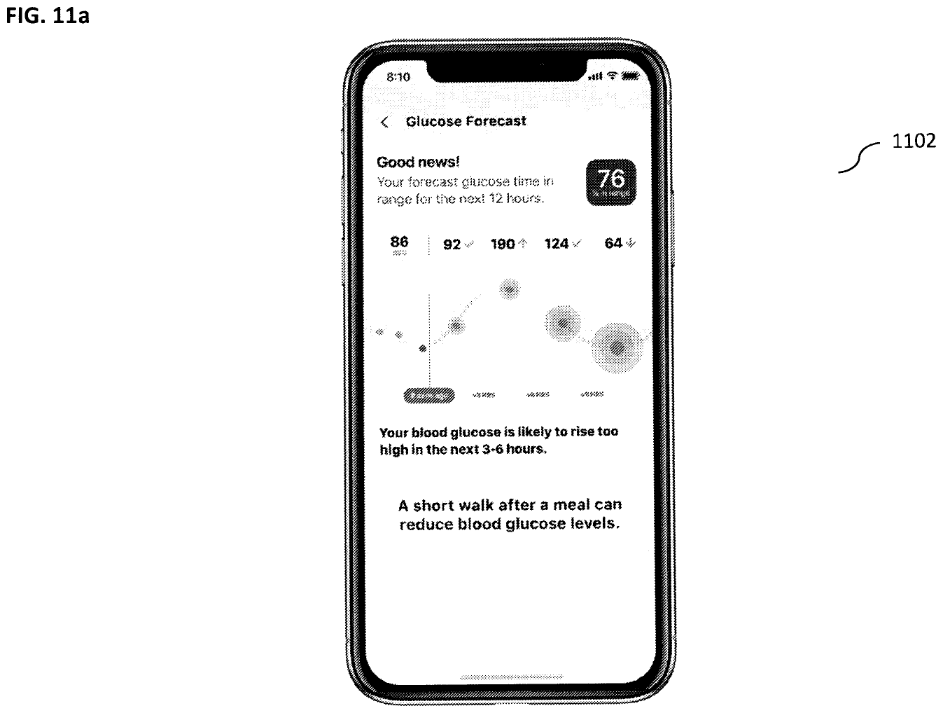

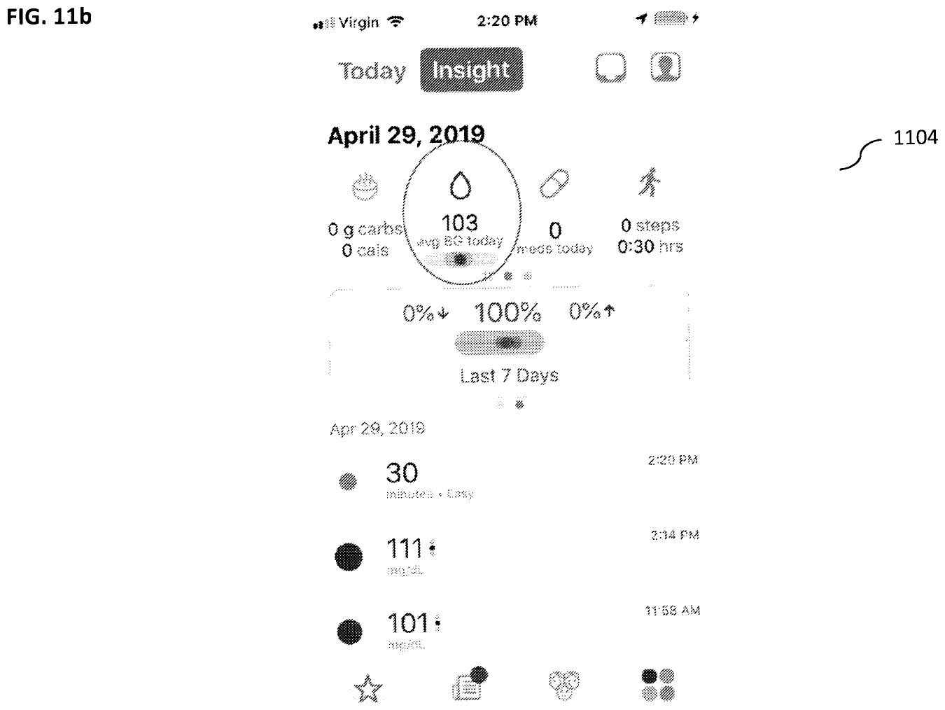

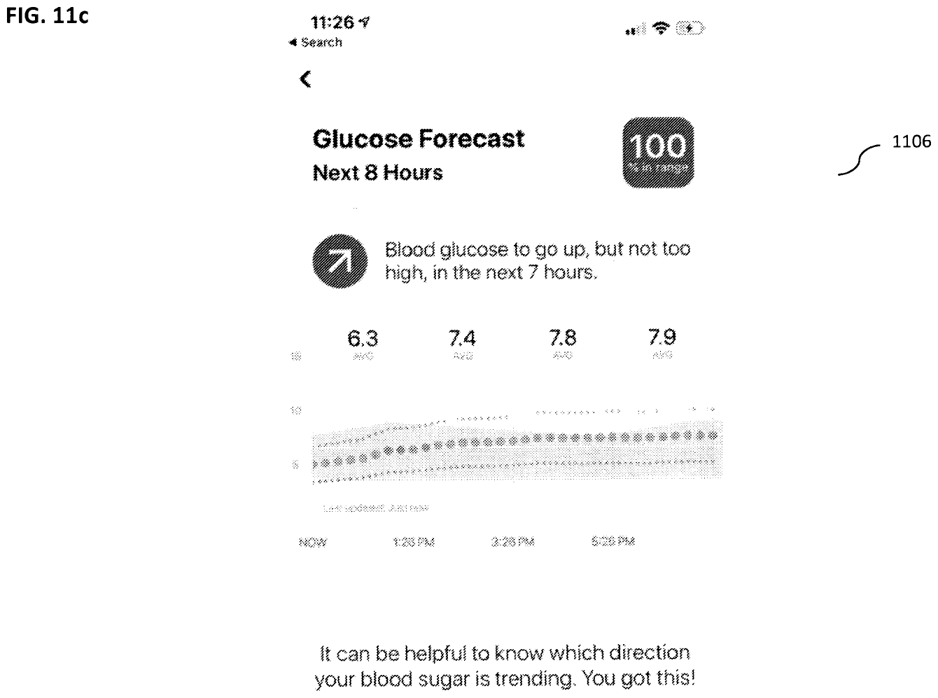

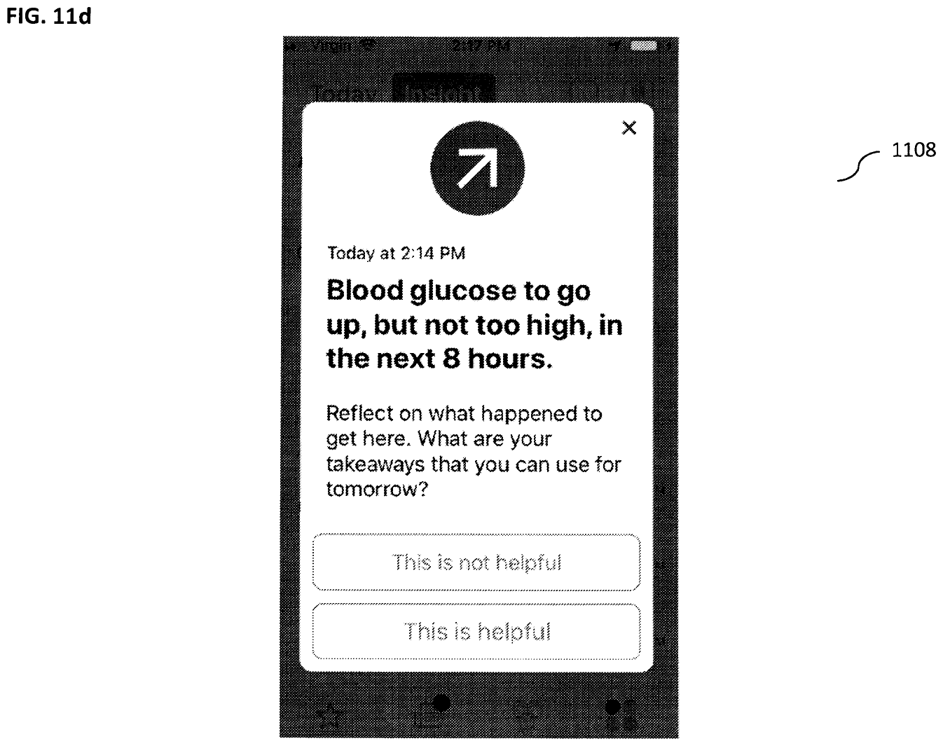

As shown in FIG. 1b, the engine 102 can include a data extraction module 112, a forecast input feature determination module 114, a trained model(s) module 116, a confidence interval forecast determination module 118, a glucose forecast determination module 120, a target band determination module 122, a forecast-in-range percentage determination module 124, a forecast summary generation module 126, and a recommendation/message generation module 128. The data extraction module 112 can be configured to obtain specific user data (e.g., data shown in FIGS. 2a-b) from the database 106 (shown in FIG. 1a) (at 111 shown in FIG. 1c). Once the user-specific data is obtained, that data can be supplied to the forecast input feature determination module 114 to determine and/or calculate forecast input forecast features from the user data (at 113 shown in FIG. 1c). The determined features can be provided to the trained models module 116 (at 115 shown in FIG. 1c). As discussed below, the trained models can be used to determine or forecast blood glucose values using the confidence interval forecast determination module 118 at any point in time in the future (e.g., in 1 minute, in 5 minutes, in 30 minutes, in 1 hour, in 5 hours, etc. and/or in any period of time) (at 117 shown in FIG. 1c). Additionally, as discussed below, the trained models can be used to determine or forecast one or more confidence intervals using glucose forecast determination module 120 (at 119 as shown in FIG. 1c). In some implementations, along with determined glucose values (determined at 117 in FIG. 1c), the user can, optionally, and/or alternatively, be presented (on the user device 104) with data that has been determined for confidence intervals (determined at 119 in FIG. 1c), target band (determined at 121 in FIG. 1c), and/or forecast-in-range percentage data (determined at 123 in FIG. 1c). Exemplary graphical user interfaces that can be generated for presentation on the user device 104 containing the above information are shown in FIGS. 11a and 11c and are discussed below.

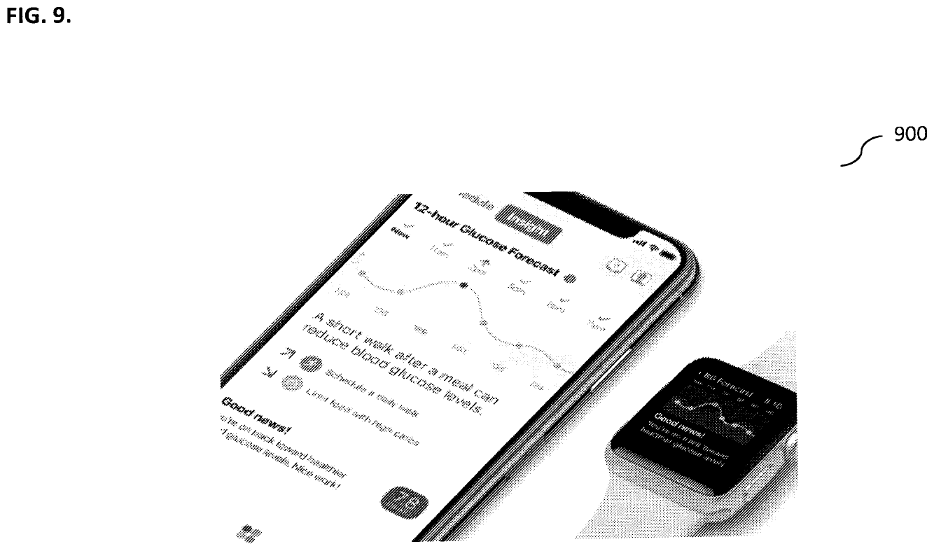

The forecasted blood glucose values can be provided to the recommendation/message generation module 128 to generate one or more recommendations for the user and/or any other important messages (at 125 as shown in FIG. 1c). Further, the forecasted blood glucose values can be provided to the forecast summary generation module 126 for the purposes of generating summary of the forecast (e.g., blood glucose is expected to rise over the next 3 hours) (at 127 as shown in FIG. 1c). The modules 126 and 128 can be configured to generate one or more graphical user interfaces (e.g., as shown in FIGS. 9-12b) and may provide various indications, suggestions, messages, etc. to the user. Along with blood glucose forecast indications, the graphical user interfaces may include one or more recommendations concerning physical exercise, diet, hydration, rest, sleep, medical appointments, reminders, and/or any other information, automatically selected to be appropriate to the user given the forecast.

In some implementations, the forecasted blood glucose values may be provided to the forecast-in-range percentage determination module 124 for the purposes of determining whether the generated forecast is in a percentage particular range of a specific target blood glucose concentration threshold value (at 123 as shown in FIG. 1c). The target band determination module 122 can be used to determine specific blood glucose concentration threshold values (at 121 as shown in FIG. 1c). The module 122 can rely on the user data (e.g., data specific to the user as well as data related to other users (e.g., including any historical data, etc.) stored in the database 106 shown in FIG. 1a) that is extracted by the data extraction module 112 from the database 106. The module 124 can perform comparison of the forecasted blood glucose concentration values (at 117 shown in FIG. 1c) to one or more threshold values determined by the module 122 (at 123 shown in FIG. 1c). Once the comparison is performed, the module 124 can be configured to generate an appropriate indication that can be displayed in one of the graphical user interfaces generated by modules 126, 128. The indication can be indicative of whether the user's blood glucose concentration is expected to fall outside of a specific threshold range, is expected to be within the threshold range, etc. The modules 126, 128 can be further configured to interpret this determination and generate one or more of the above recommendations, suggestions, indications, etc. to the user.

In some implementations, the current subject matter may also perform an assessment of accuracy of any of the above forecasted values using various techniques. For example, the forecasted values may be compared against an accuracy threshold, e.g., a standard error of prediction higher than 80 mg/dL (or any other value), or a 90% confidence interval (or any other confidence interval) of width greater than 100 mg/dL (or any other width), or a likelihood less than 75% (or any other value) of meeting any given clinical accuracy criterion such as the Clarke Error Grid zone “A” (or any other label from any error classification scheme), to determine whether the forecasted values have a high degree of uncertainty. If so, the current subject matter may determine that the forecasted values should not be delivered to the user device 104. Alternatively, the graphical user interface of the device 104 may generate an indication that the forecasted values may have a high degree of uncertainty and graphical prompt asking the user whether the user wishes to review the generated high uncertainty forecasted values, such as, for example, to ascertain where a possible error in input data values may have occurred. Once corrections, if any, are made, the forecast may be re-executed.

The process 130 can be illustrated by the following example. If the user logs a meal at 12:30 PM local time on December 23, the user device 104 can generate a request for a forecast. All of the user's logged information can be retrieved. Inputs can be computed for forecasts at 1 PM, 1:30 PM, 2 PM, 2:30 PM and so on. The trained model can then be used to predict the user's blood glucose concentration at those upcoming times.

In some implementations, referring back to FIG. 1a, the database 106 can store a plurality of tables that can be accessed through the query generated by the engine 102. The tables can store different types of information (e.g., one table can store blood glucose measurement data, another table can store user health data, etc.), where one table can be updated as a result of an update to another table. In some implementations, the data contained in the database 106 can be supplied and/or continuously updated by the users' providing of data/information (e.g., new data/information, updated data/information, modified data/information, etc.). In some exemplary implementations, the data can be supplied from one or more external sources (e.g., the database 106 can be configured to generate one or more queries and/or access data/information stored on one or more external databases, servers, etc.). The data stored in the database 106 can be organized in column and/or row format(s) as shown, for example, in FIGS. 2a-b.

The engine 102 can perform analysis of the obtained data (e.g., a statistical analysis, machine learning analysis, etc.) and generate a forecast of an expected blood glucose concentration for the user as well as provide an interpretation of the obtained data and/or the forecasted data, as further discussed below. The engine 102 can perform such analysis/assessment once and/or on a continuous basis, e.g., when updated data is supplied to the engine 102, the engine 102 can perform analysis and re-assessment of the previous forecast and update its previous prediction. In performing its analysis, the engine 102 can also generate additional queries to obtain further information. In some exemplary implementations, additional queries can be generated when new, updated, etc. forecast is to be generated. A new forecast can be requested whenever there are new data, when the user accesses a forecast user interface on the user device 104, etc. When a new forecast is requested (e.g., a forecast request is triggered), all of the user's information, including, but not limited to, any information that has been entered and/or obtained since any previous forecasts, can be obtained from the database 106 and used by the engine 102. In some implementations, the user device 104 can automatically supply the engine 102 with such information. Receipt of updated/additional information can generate a trigger and cause the engine 102 to execute a process associated with performing analysis/forecast/re-forecast/interpretation/etc. The updated/additional information can include, but is not limited to, blood glucose values, medication data, food intake data, physical activity data, etc. that can be actively and/or passively logged by the user device 104 and/or is actively and/or passively collected by the system 100 shown in FIG. 1a.

The following provides further details of a process performed by the engine 102 to generate a forecast of a blood glucose concentration for a user as well as provide an interpretation of the data and/or forecast for the user.

In some implementations, as stated above, the engine 102 can initiate its process with the raw data provided by user and/or other users through one or more user interfaces of one or more user devices 104 (e.g., an “app” installed on the user device 104 (e.g., a smartphone, a tablet, etc.), and use them to produce automated decision support for users. FIGS. 2a and 2b illustrate an exemplary data/information that can be stored in the database 106 and/or provided to/used by the engine 102 for the forecasting/interpretation purposes. FIG. 2a illustrates an exemplary table 202 containing many users' log data and FIG. 2b illustrates an exemplary table 204 containing user personal data. As can be understood, any other data can be provided to/used by the engine 202.

As shown in FIG. 2a, the data can be organized in one or more columns. The columns can include the user column 201 (e.g., having user identifier values “user1”, “user2”, “user3”, etc. to identify a row data for a particular user), a timestamp column 203 (e.g., indicating when a particular datatype has been recorded by the system 100), a datatype column 205 (e.g., indicating type of data that has been collected, e.g., “blood glucose” measurement, “carbohydrate” intake data, “medicine: insulin” intake data, “a1c” data, etc.), and value column 207 (e.g., corresponding to the specific datatype entered in the datatype column 205). As can be understood, the data contained in the 202 can be organized in any other desired fashion (e.g., column store, row store, column-row store, nested table, etc.) and/or can include any other desired information.

In some implementations, in addition to the data stored in table 202 shown in FIG. 2a, the current subject matter can stored user-specific personal data, as shown in table 204 shown in FIG. 2b. The user-specific personal data can include, but is not limited to, and can be organized in a column format. For example, user column 211 can include user identifiers (e.g., “user1”, “user2”, “user3”, etc.), timezone column 213 can include a time zone data (e.g., “New York”, “Lisbon”, etc.) corresponding to a location of the user and/or location where a specific data measurement, forecast, etc. may have occurred. The diabetes type column 215 can include data identifying a type of diabetes that a particular user may have (e.g., “Type 1”, “Type 2”, pre-diabetes, etc.). As can be understood, this column is not limited to the types of diabetes, and can include any other medical conditions that the user may have (alternatively, the table 204 can include additional columns listing user's medical conditions that may or may not be relevant to the forecasting of blood glucose concentration). Column 217 can include an information relating to the date when the user may have started using the processes performed by the system 100 (e.g., “sign-up” date). Column 219 can include a date when the user has been diagnosed with a particular type of diabetes (and/or other medical condition). Column 221 can include gender identification information. As can be understood, the table 204 can include any other data that may be used by the system 100 for the purposes of forecasting blood glucose concentrations for a particular user. The data stored in tables 202 and 204 can be appropriately de-identified, secured, and can be accessed upon providing of appropriate authentication credentials, tokens, etc.

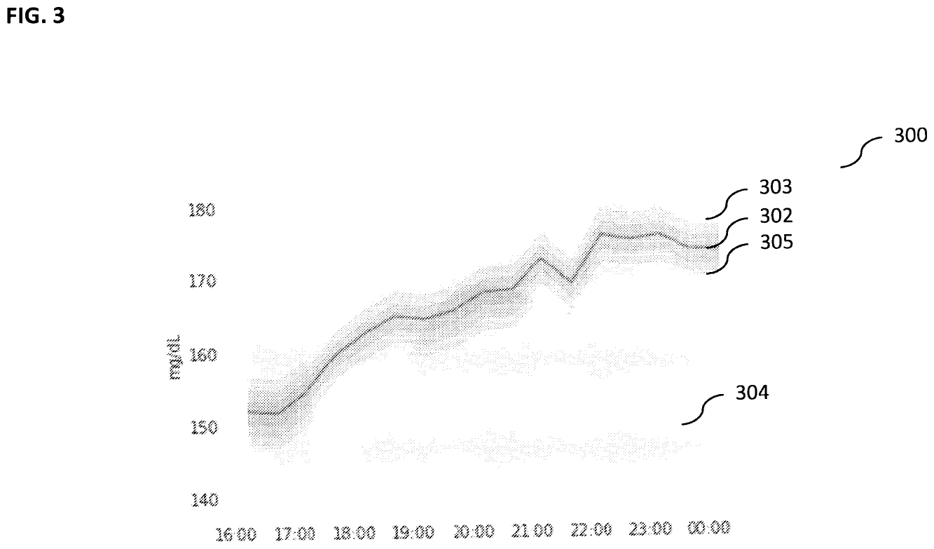

FIG. 3 is a plot 300 containing exemplary short-term blood glucose forecasts (in mg/dL over time) generated by the engine 202, according to some implementations of the current subject matter. The plot 300 contains the following three main elements: (1) an 8-to 12-hour prediction (8 shown (e.g., “16:00”, “17:00”, “18:00”, etc.) in FIG. 3) of future blood glucose concentration for a particular user (solid line 302), (2) confidence intervals for the prediction-50%, 75% and 90% intervals shown in FIG. 3 (six shaded bands 303, 305 around the solid line 302), and (3) target zone of blood glucose values associated with healthy long-term blood sugar, a1c <7% (large shaded region 304). In some implementations, this data can be provided on a graphical user interface of the device 104, which can provide an automated decision support that shows how short term forecasts can be combined with status reports (e.g., “On track”, “Off track”, etc. as referring to blood glucose concentration) as well as any personalized recommendations to the user, e.g., alter user's diet, schedule an appointment with a doctor, etc.

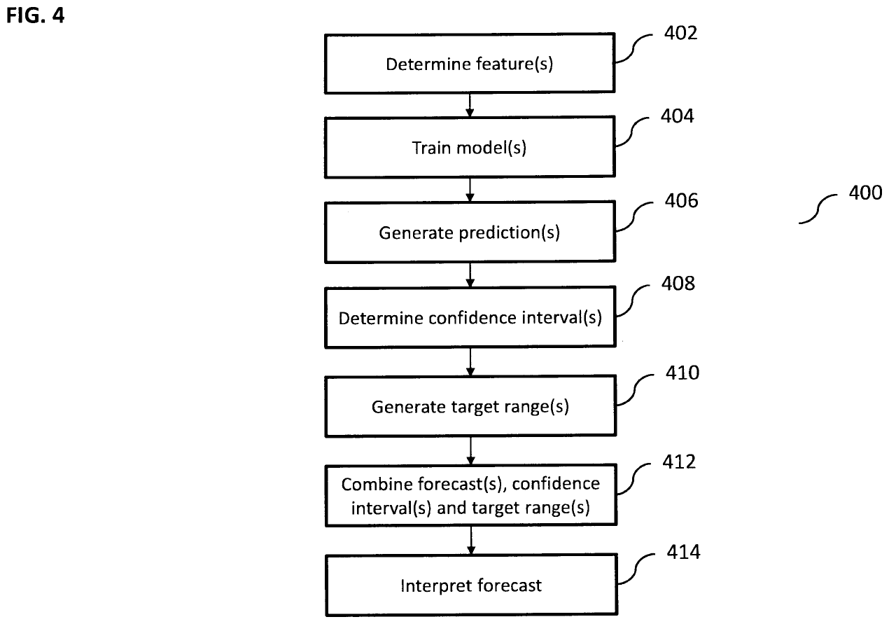

FIG. 4 illustrates an exemplary process 400 that can be performed by the engine 102 for forecasting blood glucose concentration, according to some implementations of the current subject matter. As can be understood, the process 400 can be used for any other forecasting and/or interpretation purposes. For ease of illustration, the following description will refer to forecasting and interpretation of blood glucose concentration. In some implementations, the process 400 may be executed for users that may have been diagnosed (or have the same and/or similar and/or related conditions) with Type 2 diabetes. In some exemplary implementations, the process 400 may be executed for users with any type of diabetes or any other medical conditions, e.g., users with Type 1 diabetes, users with Type 2 diabetes that are using no insulin, users with Type 2, pre-diabetes and/or gestational diabetes that are using no insulin and/or using basal insulin and/or using bolus insulin.

At 402, the engine 102 can be configured to determine features that will be used for training a forecasting data model. This can be also referred to as feature engineering. In some implementations, a large number of candidate features can be evaluated. The candidate features can be generated based on a multitude of factors and/or data. By way of non-limiting examples, the data can include data collected to date relating changes in blood glucose concentrations (e.g., whether in individuals with any type of diabetes, individuals without any type of diabetes, healthy with/out any other medical conditions, etc.) resulting from various activities (e.g., food intake, medication intake, physical activity, etc.), data specifically related to metabolic processes that cause different factors (e.g., food, medication, etc.) to affect blood glucose values. The candidate features can then be used in training and/or validation processes where a model can be trained with some of the candidate features using a first set of data (i.e., a training data), then the accuracy of that model can be evaluated by using it to predict values from a second set of data (i.e., validation data). This process can be repeated with different sets of candidate features until the features are identified that produce the best accuracy on the validation data. Because these processes can be repeated over time, specific features being used in the model can be changed and/or improved regularly.

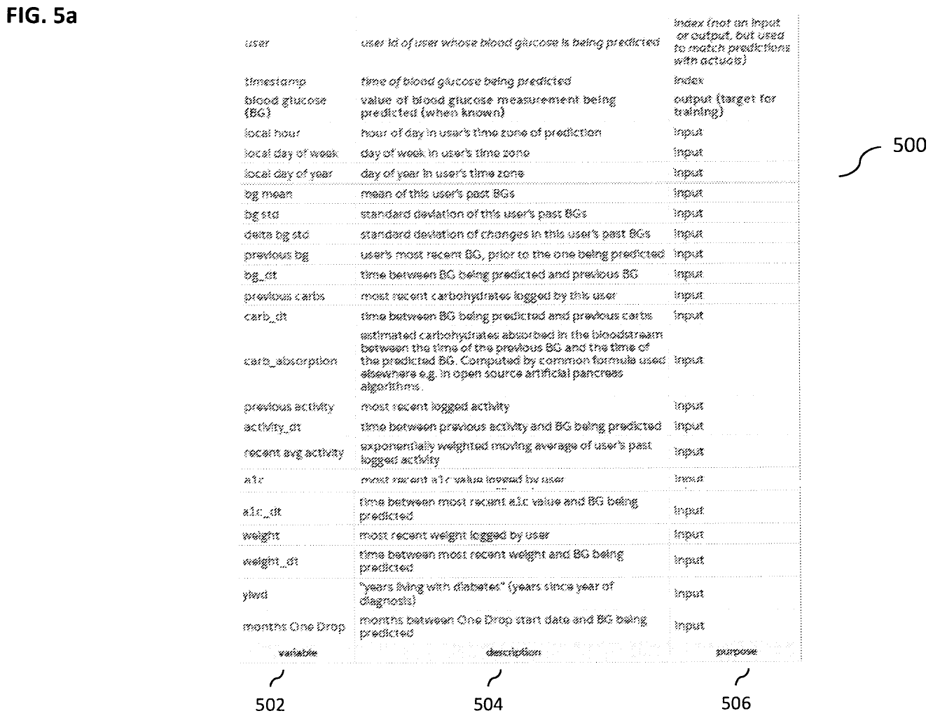

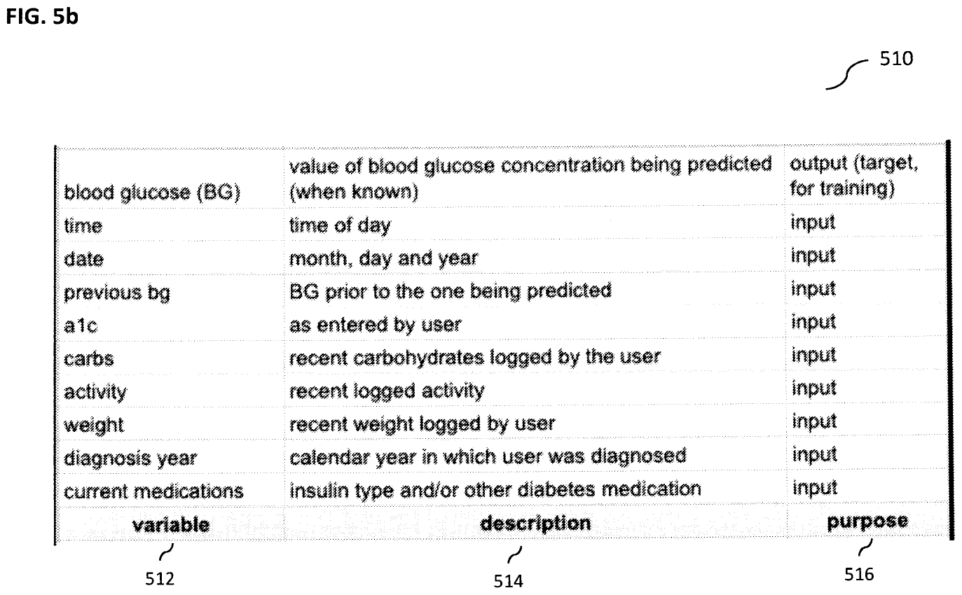

As part of the feature determination process, information shown in tables in FIGS. 2a-b can be organized into a format suitable for machine learning. The raw inputs shown in FIGS. 2a-b can be irregularly spaced in time, and different users can have different numbers of entries in the data, e.g., one user can have three blood glucose readings each day for three months, another user can have three or four per week at irregular times, etc. A user can log frequently for a while, then less frequently later. In some implementations, the current subject matter system 100 can (a) organize arbitrary past information into a common format to use as inputs for each prediction, (b) output predictions at irregular times, (c) when predicting blood glucose for one user, use relevant experience from other users, but (d) also use information particular to the user whose blood glucose is being forecast. For example, user's logged information can be expressed relative to the time being predicted and/or relative to the time of the last known value of blood glucose concentration. Tables 500 and 510, shown in FIGS. 5a-b, respectively, illustrate some examples of the features in the format used. In some exemplary implementations, each past blood glucose measurement can be set as a target to predict while using all data prior to the target measurement as input.

As stated above, FIG. 5a illustrates a table 500 containing exemplary non-limiting training model inputs (e.g., local hour, local day of week, etc.). The table 500 can include one or more columns, such as, for example, column 502 “variable” identifying specific variables being in the process 400 (shown in FIG. 4), column 504 “description” describing the variable listed in column 502, and column 506 “purpose” classifying the variables listed in column 502 (e.g., “input”, “output”, etc.). FIG. 5b illustrates a table 510, which can be a variation of table 500 shown in FIG. 5a, that includes similar columns 512-516 identifying specific variables (e.g., “time”, “date”, etc.) that can be used.

In some implementations, the current subject matter system 100 can organize the data shown in table 500 to predict blood glucose concentration at an arbitrary time, based on different filters of all previous inputs (e.g., most recent, average(s), smooth(s), metabolic action(s) since the previous blood glucose measurements, etc., and/or any combination thereof), which can be defined regardless of the number or irregularity of past data entries from a particular user. Instead of having to predict a blood glucose concentration, for example, every hour on the hour and collect training data every hour on the hour, the current subject matter system 100 can generate a prediction at any time, such as, whenever there is a blood glucose measurement and/or can use information collected prior to that time. In some implementations, for training and/or testing the forecasting model, blood glucose concentrations can be predicted based on the time they are obtained, where predictions are scored to determine how close they are to the actual concentrations.

In some implementations, the scoring can be representative of an absolute difference between predicted and actual values. Thus, smaller differences between predicted and actual can be representative of a substantially accurate prediction, a zero difference can represent a perfect prediction, etc. By way of a non-limiting example (and in addition to the discussion below), if a user recorded a blood glucose concentration (BG) of 163 mg/dL at 4 PM on Tuesday, Dec. 23, 2018, and the last information provided before that time was a meal logged at 12:30 PM on the same day, then the system 100 (shown in FIG. 1a) can predict blood glucose concentration at 4:30 PM based on all information up to 12:30 PM. Hence, if the predicted BG was 157 mg/dL, then the absolute difference can be represented by |151−163|=|−12|=12 mg/dL.

In some implementations, as the model is trained, similar examples from all users can be sorted together to provide a basis for new predictions. In some exemplary implementations, such information can be used for the purposes of making predictions for a particular user. For example, if blood glucose concentration is being predicted 12 PM on a Thursday (e.g., user local time) for a user whose previous blood glucose concentration was near 100 mg/dL, around 4 hours ago, and of a known weight, a1c, average activity, etc. then the model can predict that user's blood glucose concentration using previous examples having similar inputs from one or more other users . . . . In some exemplary, non-limiting, implementations, as stated above, only those criteria that the model training exercise discovers to be predictively useful can actually be used to determine which other users are considered “relevant”.

At 404, the system 100 can perform forecasting model training, as shown in FIG. 4. As can be understood, any known models (e.g., XGBoost, etc.) can be used for the purposes of training, testing, and/or validation using input data shown in FIGS. 5a-b. In some exemplary implementations, the model can be trained using data collected from all users up to and/or through a certain point in time, e.g., all data collected prior to September 2018. The next period of collected data can be used for validation, e.g., September 2018-March 2019. As can be understood, the current subject matter is not limited to any specific periods of time, whereby any cutoff dates can change over time as the model is trained and/or retrained periodically to encompass the growing data set.

At 406, the system 100 can generate one or more predictions of blood glucose concentration for the particular user. The trained model can be used to generate predictions in the following exemplary manner. At the time the prediction is made, input data shown in FIGS. 5a-b can be generated for specific times, e.g., at 30 minutes, at 60 minutes, at 90 minutes, etc., into the future. The data can be generated for a forecast period range of 8 to 12 hours. Other time intervals/times, can be used. For each of those forecast times, the inputs can be adjusted accordingly, so that if the prior blood glucose concentration was obtained at 8 AM, and it is now 12 PM, the forecasts can be generated for 12 PM, 12:30 PM, 1 PM, 1:30 PM, etc. Inputs appropriate to each prediction time can be generated, and this set of inputs can then be provided to the forecasting model to generate forecast blood glucose concentration values.

At 408, the system 100 can determine confidence intervals of the forecasted data. The training models used by the system 100 (e.g., XGBoost model, etc.) can include a large number of “trees” (e.g., 150). As can be understood, any training models can be used by the system 100 and the current subject matter is not limited to the XGBoost or similar such models, e.g., Gradient Boosted Trees models, etc. For ease of illustration only, the following description will refer to the models identified above. In these models, each data item can correspond to one “leaf” of each tree, and each leaf can have a “weight” that can be determined when the model is trained. The predicted value for that data item can be the sum of the weights of the leaves that that data item ends up in for each tree. In some exemplary implementations, the confidence intervals can be determined based on prediction errors/error distribution set (as discussed below). As discussed above and shown in FIGS. 1b-c, the trained model module 116 can determine one or more standard errors for the predicted blood glucose values, and can also include a table of confidence intervals as a function of a standard error. At prediction time, the trained model module 116 can generate the forecast blood glucose concentrations, and their standard errors, then determine one or more prediction confidence intervals depending on the standard errors determined by the confidence interval forecast determination module 118.

The weights can be determined as follows: each tree's contribution can be considered to be a correction to the running sum. For example, to compute weights in a leaf of a 4^{th} tree, the training routine looks at the predictions for the items in that leaf from summing their weights from the first three trees. All those 3-tree predictions can have some error, and the mean value of that 3-tree error for those items becomes the correction value, or the weight of the tree-4 leaf being computed. As such, the weight of that tree-4 leaf can be the mean value of the 3-tree errors of the items in that leaf multiplied by a value that depends on the model parameters being trained.

That weight does not consider the spread of the 3-tree errors. The errors of items in the tree-4 leaf could range from 10 to 12, with an average value of 11, or they could range from −89 to +111, with an average value of 11: the weight can be the same. However, the 4-tree prediction errors can be larger in the second case than in the first. In some implementations of the current subject matter, the system 100 can assume that if the prediction is the sum of each leaf's errors' mean, then the variance of the prediction can be the sum of the each leaf's errors' variance. The system 100 can then examine the trained model (e.g., XGBoost model) and determine the variance of errors in each leaf of each tree, and turn that into a “variance model”. The inputs to the variance model can be the same as the inputs to the prediction model: for each prediction, the variance model adds up the variances of the leaf that prediction lands in for each tree to produce the variance of the prediction. The square root of the variance gives the standard error of the prediction.

The prediction errors might not be normally distributed, however, the error distributions can be very close between training and test sets. Next, the error distribution for training set predictions with the same variance as the prediction in question can be ascertained. The quantiles of that training set distribution can then be assumed to be the quantiles of the prediction in question.

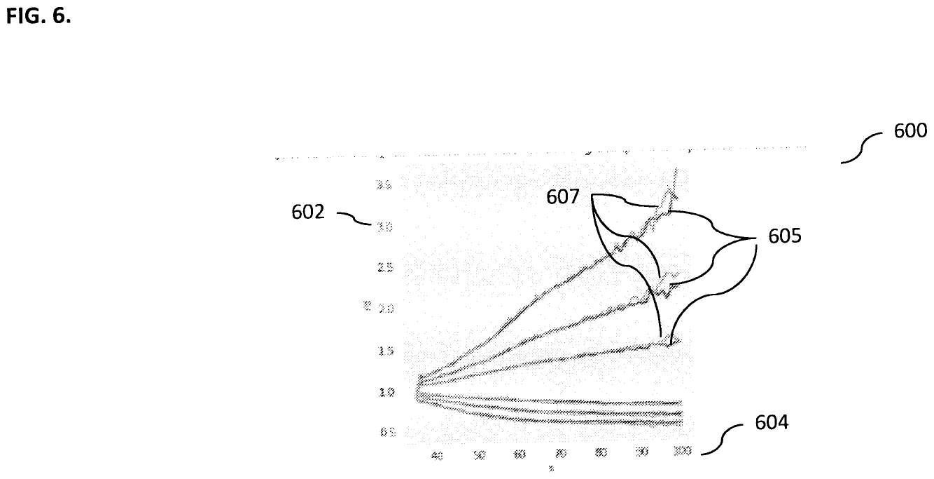

FIG. 6 is a plot 600 illustrating exemplary confidence intervals (e.g., 50%, 75%, 90%) for a training set (curves 605) and a prediction set (curves 607) (i.e., close correspondence of the training and test set quantiles). The vertical axis 602 of the plot 600 corresponds to a “relative deviation” (i.e., prediction/true value). Horizontal axis “s” 604 of the plot 600 corresponds to the standard error of each prediction. The plot 600 further shows that error distributions are strong functions of standard error of prediction and that the distributions generalize from training to test sets for any particular variance value. Once the relative deviations are known for the prediction, they can be multiplied by the predicted blood glucose concentration to get the actual confidence interval in mg/dL.

Referring back to FIG. 4, at 410, one or more target ranges can be generated. In some exemplary implementations, a user can identify upper and lower limits that the user desires to stay between, e.g., between 70-140 or 70-170 mg/dL. As can be understood, other values can be used.

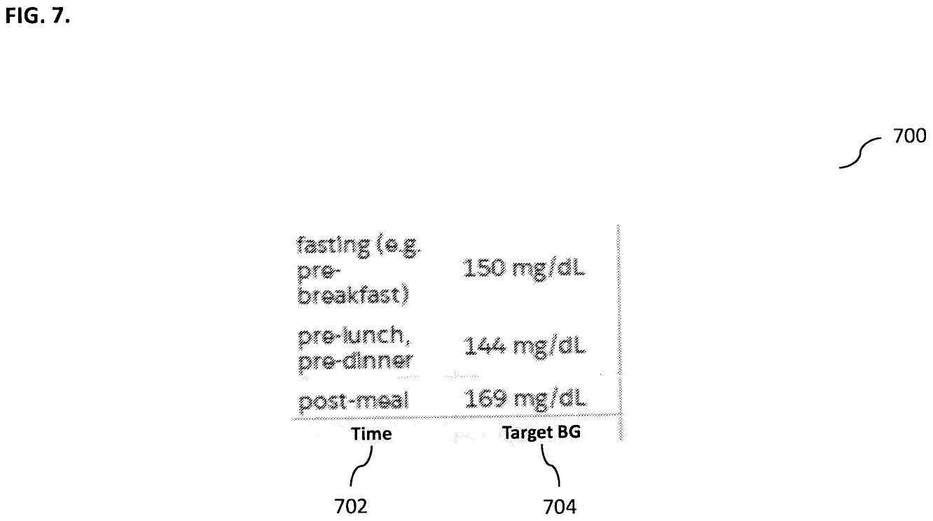

This information can be used to help the user to interpret forecasted blood glucose concentration in terms of whether the forecast was in line with healthy a1c values, above, below, etc. Unlike constant values, blood glucose target ranges shift throughout the day, depending on when the user eats, performs various activities, etc. To develop a target range (e.g., as shown in FIG. 3, the large shaded region 304), various values can be used, such as those shown in table 700 of FIG. 7.

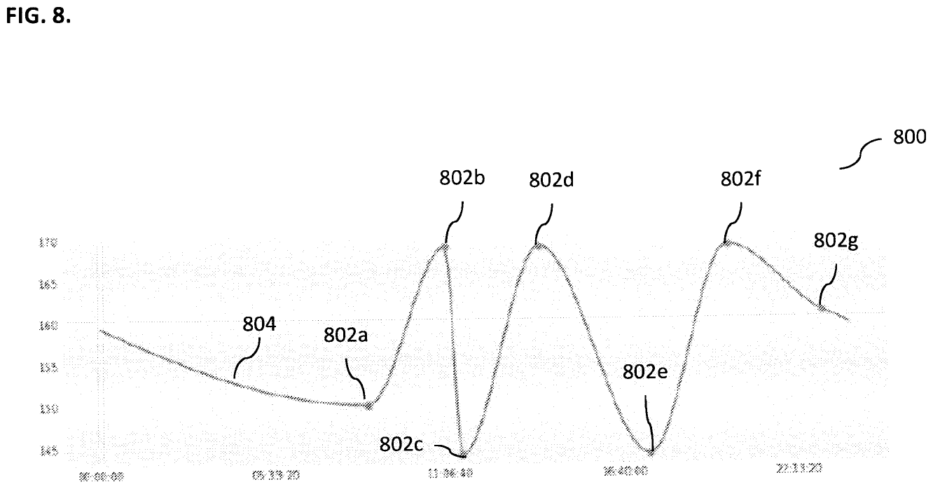

The values in table 700 (in column time 702 and column “target BG”) 704) can correspond to upper ends of ranges of blood glucose concentration readings from users who had a1c levels under 7%. To translate those to times of day, observed meal times data in the log for a particular user can be used. In some exemplary implementations, logged carbohydrate values can be tagged as “breakfast”, “lunch”, or “dinner.” Data from 30 minutes before meals can be treated as “pre-meal” data, 2 hours after meals as “post-meal” data, and late evening data as “bedtime” data. If a user has not logged a meal during a past period, or if target ranges are being determined for future hours when meals have not happened yet, user's most frequent meal time can be used for that meal. If the user has not logged at least 3 meals of that meal type, the system 100 can assume the most popular meal times using data obtained from other users. Once the meal times are determined for a particular day, target points at correct pre-meal and post-meal times can be plotted, as shown by the plot 800 in FIG. 8. These can be interpolated using a standard interpolation (e.g., pchip interpolation). As shown in FIG. 8, target blood glucose concentration values (mg/dL, vertical axis) can placed at specific times (shown as dots 802 (a, b, c, d, e, f, g)) based on user-specific meal times. Values between dots can be interpolated (shown by the solid line 804 in FIG. 8).

Referring back to FIG. 4, at 412, forecast data, confidence interval(s), and target range(s) can be combined into a single plot (e.g., as shown in FIG. 3 and can be displayed on a user interface of a user device, as shown in FIG. 9). This can inform the user of likely near-term blood glucose concentration values and their uncertainties, provide a useful reference for comparison, and can allow the user to make decisions about whether or not to change plans and/or take any action (e.g., if a forecast is going from 130 to 150, it may or may not be acceptable and may depend on meal time(s)).

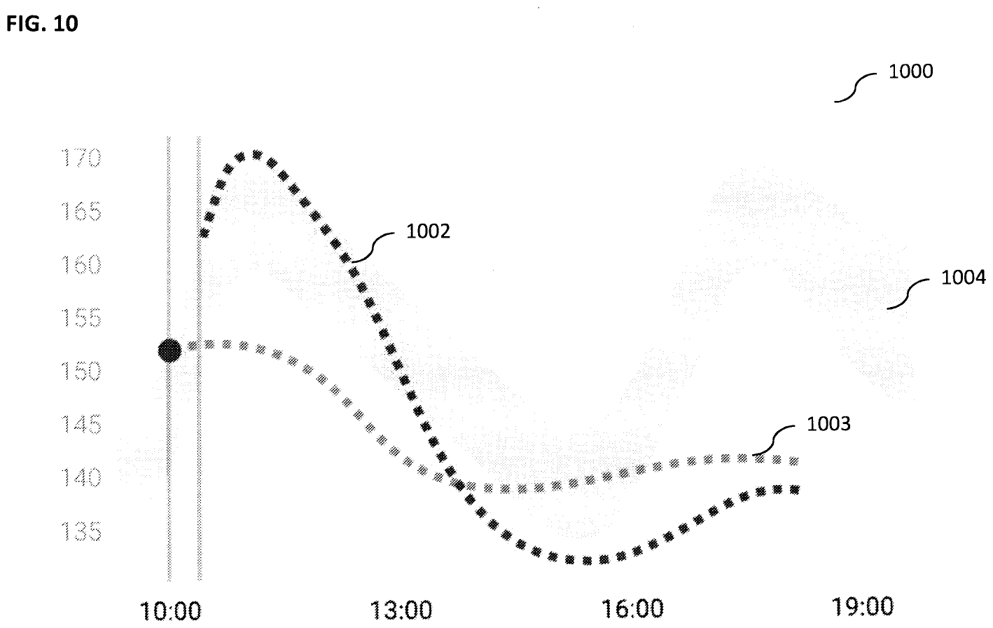

At 414 of FIG. 4, the system 100 can interpret forecasts. An exemplary forecasting plot 1000 is shown in FIG. 10. In particular, the system 100 can compare the forecast blood glucose concentration values 1002, 1003 to the target range 1004 values at forecast times. If more than a threshold percentage (10% or 25%) of the forecast points are above the target range, the forecast can be labeled “high”. The system 100 can generate a message for display to the user, which can state, for example, “Likely to go higher than recommended within 4 hours”, “Likely to remain within healthy levels for the next 8 hours”. That determination is also used as an input to automatically select a support message that can provide the user with various actions that the user can undertake.

In some exemplary implementations, support messages can be in one or more of the following categories (or any other categories):
advice/education for when user's forecast is higher than target range. These messages can contain advice about eating, exercise, sleep, stress and many other factors that affect blood glucose.
encouragement/celebration for when user's forecast is within target range. These messages can contain positive feedback to re-enforce user behaviors that lead to healthy glucose levels.

In some implementations, based on the forecast interpretation, the user device 104 can display a message (e.g., as shown in FIG. 9) using one or more of the above or any other categories. In some implementations, users can be offered an opportunity to mark support messages as useful/not useful, based on which, subsequent messages to users can tailored to be most helpful to each particular user.

FIGS. 11a-d illustrate various exemplary graphical user interfaces 1102-1108 that may be generated by the current subject matter system 100 shown in FIG. 1. The user interfaces 1102-1108 may be generated during execution of and/or after completion of the process of forecasting blood glucose concentration (e.g., process 400 shown in FIG. 4). The user interfaces 1102-1108 may be displayed on any of user's computing devices, smartphones, tablets, personal computers, laptops, smart watches, etc. The arrangement and/or formatting of displayed information may be tailored to such specific devices, where the current subject matter may determine the type of computing device that the user is using and graphically format the information that is to be displayed to the user. Such formatting may include displaying different graphical elements (e.g., buttons, pointers, colors, headings, tabs, tables, etc.) as well as their arrangement on a display screen.

FIG. 11a illustrates an exemplary graphical user interface 1102 that may be used to provide an addition means for interpretation of a blood glucose concentration forecast. In some exemplary implementations, the interface 1102 may be configured to display a “score” associated with the forecast along with various values for blood glucose concentration (e.g., “92”, “190”, “123”, “64”, etc.) over a period of time and/or determined at particular periods of time, as for example, determined by the process 400 shown in FIG. 4. The interface 1102 may also display an average blood glucose concentration (e.g., “86”) as well as whether blood glucose concentrations are higher or lower (as shown by the up and down arrows) or normal (as shown by a check mark). The score displayed by the interface 1102 (i.e., “76”) may correspond to a 76% of time that the blood glucose concentration is forecast to be in a “normal” range. Further, the interface 1102 may also alert the user, based on the execution of the process 400, that the user's blood glucose levels are likely to rise to higher levels and may display various suggestions for reducing blood glucose levels, e.g., “A short walk after a meal can reduce blood glucose levels.” Such messages may be retrieved from memory upon determination of the specific BG values, score, historical values, values ascertained through machine learning, and/or any other information.

FIG. 11b illustrates an exemplary graphical user interface 1104 that may be used to provide “insight” to the user. For example, the interface 1104 may indicate a specific average BG (e.g., “103”) for a particular day (e.g., “Apr. 29, 2019”). It may also provide the user with information for specific BG concentration at particular times (e.g., “111 mg/dL” at 2:14 PM, etc.). The interface 1104 may display user's exercise minutes, information about meals consumed along with amount of carbohydrates, fats, etc. contained in those meals, medications taken, etc. Further, the interface 1104 may include information about how user's BG concentration may have changed, has changed, will change during a particular period of time. (e.g., “Last 7 days”). The user interface 1104 may be customized by the user, as desired.

FIG. 11c illustrates another exemplary user interface 1106 that may be generated by the current subject matter. The interface 1106 may be configured to display to the user a graphical plot containing predicted blood glucose concentration over a period of time (e.g., “Next 8 Hours”). It may also display a specific conclusion concerning predicted blood glucose concentration, e.g., “Blood glucose to go up, but not too high, in the next 7 hours”. Average blood glucose concentration values (now and predicted) may be displayed as well (e.g., “6.3”, “7.4”, “7.8”, “7.9” mmol/L). The plot shown in the interface 1106 may illustrate how the values may change over time along with any error margins and a normal range.

FIG. 11d illustrates yet another exemplary user interface 1108, which may be used by a user to provide feedback to the system 100 regarding information that is being provided to the user in connection with blood glucose concentration predictions. The user interface 1108 may display a specific message “Blood glucose to go up, but not too high, in the next 8 hours. Reflect on what happened to get here. What are your takeaways that you can use for tomorrow?” as well as voting buttons “This is not helpful”, “This is helpful”. As can be understood, other messages, buttons, feedback windows, and/or elements may be displayed to the user. By clicking or pressing one of the buttons, the user may submit a feedback to the system 100 (e.g., via a communication message that may be specifically formatted and transmitted for interpretation by the system 100 shown in FIG. 1a). Further, as can be understood, other graphical user interfaces and/or elements may be displayed to the user to provide information concerning blood glucose concentration prediction, analysis, suggestions, etc.

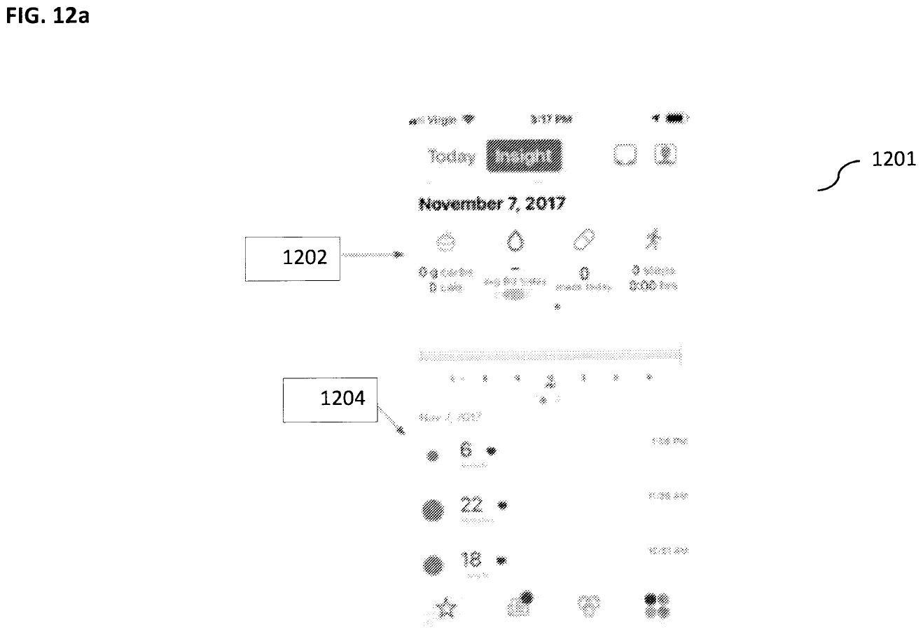

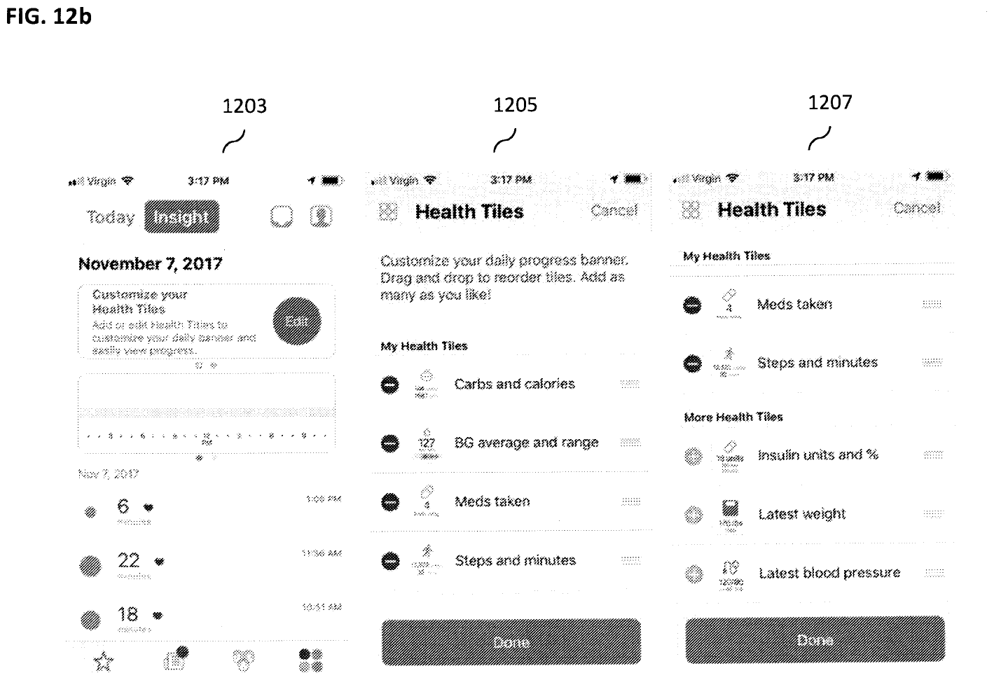

FIGS. 12a-b illustrate exemplary user interfaces 1201-1207 that may be used by the user to customize information that is being displayed to the user. As stated above, the interfaces may be part of an application (“app”) that may be available on user's computing devices. The “app” may be configured to allow collection, management and/or use of health data that may be related to various medical conditions, including but not limited to, diabetes, hypertension, hyperlipidemia, and/or any other conditions and/or any combinations thereof. The “app” may collect and/or analyze various types of data that may include, but is not limited to, medications, food, physical activity, weight, blood glucose concentration, blood pressure, and any other type of data.

In some implementations, the user interfaces may be configured to allow user to customize or personalize display of various health related and/or any other data through use of various graphical user interface elements, which may include but are not limited to, buttons, screens, tiles, pointers, etc. (hereinafter, “tiles”). The tiles may allow users to customize/personalize the display of health data across multiple conditions. By way of a non-limiting example, the tiles may enable users to combine data from manual user input and/or automatic data ingestion from multiple information sources, view key health information at a glance across multiple conditions, perform deeper analysis of user's data along with various health metrics, customize user interface display to their preference, etc.

FIG. 12a illustrates an exemplary user interface 1201. The user interface 1201 may include tiles 1202 (e.g., four health tiles are shown: Food, Glucose, Medications, and Activity). Each tile 1202 may include a graphic and/or metric summarizing the user's data for a particular time period (e.g., a day). The user may click on a tile to populate a lower part 1204 of the screen with additional and/or more in-depth information. In the user interface 1201, the lower part 1204 illustrates a history of activity-related data are shown, but the nature of the in-depth view may depend on the tile selected. For example, the Glucose tile may show a forecast of expected glucose values over a particular time period (e.g., the upcoming day). The Water Intake or Food tile may show an estimate of the user's hydration level.

FIG. 12b illustrates user interfaces 1203-1207 that may be used for the purposes of customizing displays of information. The user interfaces 1203-1207 may be accessed by the user by pressing a particular graphical user interface element on one or more prior screens, “swiping” (e.g., left to right, right to left, up to down, down to up, etc.). For example, by swiping the tiles to the right on user interface 1201, the user may reveal an option to configure tiles (e.g., as shown by interface 1203) and may choose any and/or all of available tiles and order them as desired. For example, the user may choose to edit tiles that may relate to user's data related to food, medications, activity, glucose, blood pressure, weight, and A1C (e.g., a laboratory measure of long-term blood glucose concentration), cholesterol, self-care habits, hydration, etc. Activating customization mode on the user interface 1203, the user may be prompted to user interfaces 1205-1207, where the user may choose to remove and/or add display of certain information (e.g., by tapping “−” or “+” signs, respectively).

### EXEMPLARY EXPERIMENTAL IMPLEMENTATIONS

The following provides a discussion of an exemplary experimental implementation of system 100 (as available from Goldner, D. R., “A Machine-Learning Model Accurately Predicts Projected Blood Glucose,” Diabetes 2018July; 67 (Supplement 1)). In this experimentation, 1,923,416 BG measurements from 14,706 people with noninsulin treated Type 2 diabetes were collected. Additionally, various contextual information (CI) was collected. The CI included at least one of the following/various combinations of the following: demographic data, health data (e.g., weight, A1c), etc.

Input to the prediction model: prior blood glucose data and CI.

The model did not distinguish between users' BGs with similar CI. Forecast horizons were determined using time since prior blood glucose concentrations and varied from 10 minutes to several days. Machine learning algorithms to predict BG values were trained and tested on BGs entered prior to September 2017 (83% of all BGs). BGs (17%) entered from September-November2017 were held out and predicted.

Results: Users were 59% male, with 80% from North America, 9% from Europe, and 11% from elsewhere. 50% of users were diagnosed with Type 2 diabetes in the past 3 years. The median and mean absolute error of holdout predictions were 14.2 and 21.3 mg/dL respectively, with 91% of predictions within +/−50 mg/dL.

In another exemplary experimental implementation, an initial sample of 23,876 forecasts were sent via in-app notifications to 4,679 users with Type 2 diabetes. Forecasts included of BG trend, duration, and level (“rising, but not too high, over the next 3 hours”) and, when appropriate, a support message relevant to forecast and user history. Forecast delivery was random, triggered with 50% probability when information was logged, no more than once/day/user.

Forecasts could be rated “Useful” or “Not Useful”. A machine learning model, trained on the initial sample, predicted the probability of each type of feedback. A second sample of 28,838 forecasts were sent to 5,506 users, with probability of usefulness determined by the model.

Results: in the first sample, 42.8% of forecasts received feedback from 69.6% of users; 87.1% was “Useful”. In the second sample, 63.7% of forecasts received feedback from 67.1% of users; 92.4% was “Useful”.

The experimental results demonstrated that a new machine learning model tailored forecast delivery, reducing the “Not Useful” rate by 41.1% (from 12.9% of feedback to 7.6%).

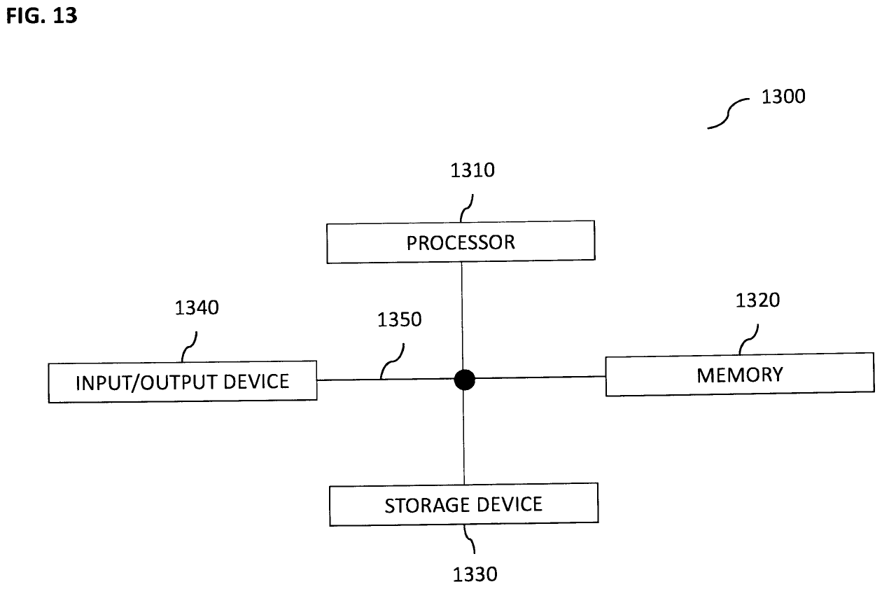

In some implementations, the current subject matter can be configured to be implemented in a system 1300, as shown in FIG. 13. The system 1300 can include a processor 1310, a memory 1320, a storage device 1330, and an input/output device 1340. Each of the components 1310, 1320, 1330 and 1340 can be interconnected using a system bus 1350. The processor 1310 can be configured to process instructions for execution within the system 1300. In some implementations, the processor 1310 can be a single-threaded processor. In alternate implementations, the processor 1310 can be a multi-threaded processor. The processor 1310 can be further configured to process instructions stored in the memory 1320 or on the storage device 1330, including receiving or sending information through the input/output device 1340. The memory 1320 can store information within the system 1300. In some implementations, the memory 1320 can be a computer-readable medium. In alternate implementations, the memory 1320 can be a volatile memory unit. In yet some implementations, the memory 1320 can be a non-volatile memory unit. The storage device 1330 can be capable of providing mass storage for the system 1300. In some implementations, the storage device 1330 can be a computer-readable medium. In alternate implementations, the storage device 1330 can be a floppy disk device, a hard disk device, an optical disk device, a tape device, non-volatile solid state memory, or any other type of storage device. The input/output device 1340 can be configured to provide input/output operations for the system 1300. In some implementations, the input/output device 1340 can include a keyboard and/or pointing device. In alternate implementations, the input/output device 1340 can include a display unit for displaying graphical user interfaces.

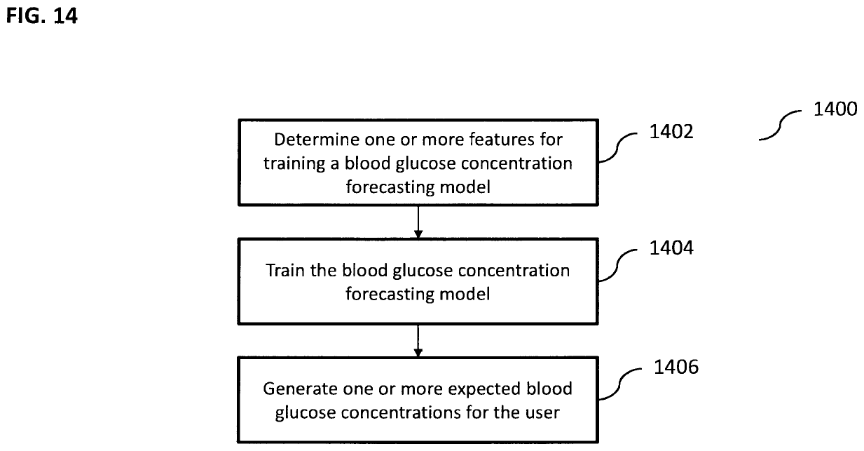

FIG. 14 illustrates an exemplary process 1400 for forecasting blood glucose concentration, according to some implementations of the current subject matter. At 1402, one or more features for training a blood glucose concentration forecasting model can be determined. The features can be determined based on one or more input data parameters associated with a user in a plurality of users. At 1404, using the determined features, the blood glucose concentration forecasting model can be trained. At 1406, using the trained blood glucose concentration forecasting model, one or more expected blood glucose concentrations for the user can be generated.

In some implementations, the current subject matter can include one or more of the following optional features. The method can further include displaying the generated expected blood glucose concentrations for the user on one or more graphical user interfaces.

In some implementations, the training can include training the blood glucose concentration forecasting model using one or more parameters associated with one or more other users in the plurality of users. The parameters associated with other users can include one or more historical data parameters associated with one or more other users.

In some implementations, the input parameters can include at least one of the following: a data indicating a type of diabetes of the user, a data indicating a medical condition of the user, a data indicating medication being consumed by the user, a data indicating a meal consumed by the user, a data indicating a physical activity performed by the user, a data indicating a time of a blood glucose concentration measurement of the user, a data indicating at least one of a previous and a current value of a blood glucose concentration measurement of the user, a data indicating a time of a previous blood glucose concentration forecast, a data indicating a target blood glucose concentration (a1c) for the user, a data indicating at least one of a current date and a current time, a data indicating a weight of the user, a data indicating one or more changes in the blood glucose concentration of the user, a data indicating one or more carbohydrate values as consumed by the user, and any combination thereof.

In some implementations, the generating can include generating one or more target blood glucose concentration ranges for the user, generating one or more confidence intervals for the generated expected blood glucose concentrations, where the confidence intervals can indicate an accuracy of the generated one or more expected blood glucose concentrations, and comparing the generated target blood glucose concentration ranges, the confidence intervals for the generated expected blood glucose concentrations, and the generated expected blood glucose concentrations. The method can also include displaying, based on the comparison, an indication whether the generated expected blood glucose concentrations is within the target blood glucose concentration ranges. The method can further include generating an alert to the user when the generated expected blood glucose concentrations is not within the target blood glucose concentration ranges.

In some implementations, the generated expected blood glucose concentrations can be generated for a point in time subsequent to the determining.

In some implementations, the method can also include repeating the determination of the features, as well as the training of the forecasting model, and then, generating, based on the repeated determinations and training, one or more updated expected blood glucose concentrations for the user.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

The systems and methods disclosed herein can be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

The foregoing description is intended to illustrate but not to limit the scope of the invention, which is defined by the scope of the appended claims. Other implementations are within the scope of the following claims.

These computer programs, which can also be referred to programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The subject matter described herein can be implemented in a computing system that includes a back-end component, such as for example one or more data servers, or that includes a middleware component, such as for example one or more application servers, or that includes a front-end component, such as for example one or more client computers having a graphical user interface or a Web browser through which a user can interact with an implementation of the subject matter described herein, or any combination of such back-end, middleware, or front-end components. The components of the system can be interconnected by any form or medium of digital data communication, such as for example a communication network. Examples of communication networks include, but are not limited to, a local area network (“LAN”), a wide area network (“WAN”), and the Internet.

The computing system can include clients and servers. A client and server are generally, but not exclusively, remote from each other and typically interact through a communication network. The relationship of client and server arises by virtue of computer programs running on the respective computers and having a client-server relationship to each other.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

1-30. (canceled)

31. A computer-implemented method associated with a user device of a user, comprising:
determining one or more training features for training a blood glucose concentration forecasting machine learning model that is specific to the user, wherein determining the one or more training features includes:
identifying a plurality of candidate features based on input data parameters associated with a plurality of other users;
training a general blood glucose concentration forecasting machine learning model using a first portion of the input data associated with the plurality of other users;
evaluating the general blood glucose concentration forecasting machine learning model using a second portion of the input data associated with the plurality of other users to identify a subset set of the candidate features that causes the general blood glucose concentration forecasting machine learning model to have a best accuracy when the general blood glucose concentration forecasting machine learning model is applied to the second portion of the input data;
receiving, from a user device, input data parameters from a user device of the user; and
identifying the one or more training features in the received input data parameters received from the user based on the identified subset set of the candidate features;
training the blood glucose concentration forecasting machine learning model using at least a portion of the input data parameters associated with the plurality of other users that is associated with the determined one or more training features; and
inputting the input data parameters received from the user device into the blood glucose concentration forecasting machine learning model to generate one or more expected blood glucose concentrations for the user.

32. The method according to claim 31, further comprising displaying the generated one or more expected blood glucose concentrations for the user on one or more graphical user interfaces of the user device.

33. The method according to claim 31, wherein the one or more input data parameters associated with the plurality of other users include a plurality of irregularly spaced historical data parameters associated with one or more of the plurality of other users.

34. The method according to claim 31, wherein the one or more input parameters received from the user include at least one of the following: a data indicating a type of diabetes of the user, a data indicating a medical condition of the user, a data indicating medication being consumed by the user, a data indicating a meal consumed by the user, a data indicating a physical activity performed by the user, a data indicating a time of a blood glucose concentration measurement of the user, a data indicating at least one of a previous and a current value of a blood glucose concentration measurement of the user, a data indicating a time of a previous blood glucose concentration forecast, a data indicating a target blood glucose concentration (a1c) for the user, a data indicating at least one of a current date and a current time, a data indicating a weight of the user, a data indicating one or more changes in the blood glucose concentration of the user, a data indicating one or more carbohydrate values as consumed by the user, and any combination thereof.

35. The method according to claim 31, further comprising:
generating one or more target blood glucose concentration ranges for the user;
generating one or more confidence intervals for the generated one or more expected blood glucose concentrations, the confidence intervals being indicative of an accuracy of the generated one or more expected blood glucose concentrations; and
comparing the generated one or more target blood glucose concentration ranges, the one or more confidence intervals for the generated one or more expected blood glucose concentrations, and the generated one or more expected blood glucose concentrations.

36. The method according to claim 35, further comprising displaying, based on the comparison, an indication whether the generated one or more expected blood glucose concentrations is within the one or more target blood glucose concentration ranges.

37. The method according to claim 36, further comprising generating an alert to the user when the generated one or more expected blood glucose concentrations is not within the one or more target blood glucose concentration ranges.

38. The method according to claim 31, wherein the generated one or more expected blood glucose concentrations is generated for a point in time subsequent to the determining.
